(19) United States
(12) Patent Application Publication
(10) Pub. No.: US 2025/0135069 A1
(43) Pub. Date: May 1, 2025

(54) APPARATUS AND METHODS OF A TUBULAR TISSUE AS A THERAPEUTIC AGENT-PRODUCING SHEATH FOR A VASCULAR GRAFT

(71) Applicant: Humacyte, Inc., Durham, NC (US)

(72) Inventors: Mehmet Hamdi KURAL, Cary, NC (US); Laura Elizabeth NIKLASON, Greenwich, CT (US)

(21) Appl. No.: 18/937,529

(22) Filed: Nov. 5, 2024

Related U.S. Application Data
(63) Continuation of application No. PCT/US2023/068296, filed on Jun. 12, 2023.
(60) Provisional application No. 63/503,137, filed on May 18, 2023.
(60) Provisional application No. 63/351,677, filed on Jun. 13, 2022.

(51) Int. Cl. A61L 27/50; A61F 2/852; A61L 27/36; A61L 27/38; A61L 27/52

(52) U.S. Cl. CPC A61L 27/507; A61F 2/852; A61L 27/3633; A61L 27/3891; A61L 27/52; A61L 2430/34

(57) ABSTRACT

System, apparatus, and method for replacing a segment of vasculature, tissue, or organ.

### CROSS-REFERENCE TO RELATED APPLICATIONS

This application is a continuation of International Application No. PCT/US2023/068296, filed Jun. 12, 2023, which claims the benefit of U.S. Provisional Patent Application No. 63/351,677, filed on Jun. 13, 2022, and U.S. Provisional Patent Application No. 63/503,137, filed on May 18, 2023, the content of each of which is herein incorporated by reference in its entirety.

### FIELD OF THE DISCLOSURE

The present disclosure relates to tissue-engineered vascular grafts.

### BACKGROUND

Tissue damage, dysfunction, or loss is a feature of a wide variety of medical conditions. Atherosclerosis, in which formation of fatty plaques in blood vessel walls leads to narrowing of the vessels, is one well-known example. Accidents frequently result in damage to tendons, ligaments, and joints. Degenerative diseases such as arthritis represent another source of injury to such tissues. Systemic diseases such as diabetes, cancer, and cirrhosis are yet another cause of organ destruction or dysfunction.

In many of the situations described above, replacement of the damaged tissue or organ is the best or even the only option. Transplantation from human donors (either live or cadaveric) has enjoyed significant success, and procedures such as liver, heart, and kidney transplants are becomingly increasingly common. However, the severe shortage of donors, the complexity of harvesting organs and delivering them to the recipient, and the potential for transmission of infectious agents are significant shortcomings of this approach. In some situations, such as replacement of blood vessels, vessels are removed from one portion of the body and grafted elsewhere to bypass sites of obstruction. However, the number of available vessels is limited, and those available may not be optimal in terms of strength or other parameters.

Use of synthetic materials or tissues derived from animals offer alternatives to the use of human tissues. For example, grafts made of synthetic polymers such as Dacron® find use in the replacement of vessels. Mechanical prostheses are widely used to replace damaged heart valves. However, use of synthetic materials has a number of disadvantages. Frequently the material is immunogenic and can serve as a nidus for infection or inflammation. Use of animal tissues also poses problems of immunogenicity as well as the potential to transmit diseases. In addition, harvested animal tissues may be suboptimal in terms of size, shape, or other properties, thus limiting the utility and flexibility of this approach. There is a need for innovative approaches to the problem of replacing damaged or dysfunctional organs and tissues.

Tissue engineering seeks to develop techniques for culturing replacement tissues and organs in the laboratory. The general strategy for producing replacement tissues utilizes mammalian cells that are seeded onto an appropriate substrate for cell culture. The cells can be obtained from the intended recipient (e.g., from a biopsy), in which case they are often expanded in culture before being used to seed the substrate. Cells can also be obtained from other sources (e.g., established cell lines). After seeding, cell growth is generally continued in the laboratory and/or in the patient following implantation of the engineered tissue. In certain cases, the replacement tissue is decellularized immediately prior to implantation.

Tissue engineered constructs may be used for a variety of purposes including as prosthetic devices for the repair or replacement of damaged organs or tissues. Of particular interest are vascular tissue-engineered constructs. There are 1.4 million surgical procedures performed annually in the United States that require arterial prostheses. Small arteries with diameters less than five to six mm cannot be replaced with artificial materials due to high rates of thrombosis. Thus, autologous vein or artery grafts are generally used to replace small arteries in the coronary or peripheral circulations. Venous grafts have thin walls that are sometimes damaged when transplanted into the arterial system, and suitable veins are not available in all patients due to amputation or previous vein harvest. Internal mammary arteries, which comprise the majority of arterial grafts, are useful only in the coronary circulation. The availability of tissue-engineered constructs for vascular applications, therefore, would fill an immediate and pervasive need.

The drug-producing cell/fibrinogen/CaCl/thrombin mixture can be casted onto Pluronic treated rectangular molds to create drug-producing cell-populated fibrin sheets. In some approaches these drug-producing cell-populated fibrin patches can be reinforced with biodegradable polymer meshes. Alternatively, the drug-producing cell-populated fibrin patches can have collagenous edges to suture onto tendons or other connective tissues.

Though full of promise, the implementation of tissue-engineered vascular grafts has been difficult. Regarding cell-laden tissue-engineered vascular grafts, and despite the recent advancements in pre-vascularization of the tissue engineered constructs, cells in these constructs quickly die due to the limited capacity for anastomoses between the vascular network of the implanted tissue and the vasculature of the recipient. Therefore, the present disclosure provides systems and methods to address viability concerns associated with tissue-engineered vascular grafts.

### BRIEF SUMMARY

The present disclosure relates to a system, apparatus, and method for replacement of a segment of vasculature.

In embodiments, the present disclosure further relates to a system for replacement of a segment of vasculature, comprising a tubular vascular graft concentrically arranged against an outer surface of a first mandrel, and a tubular support graft concentrically arranged against an outer surface of a second mandrel, the tubular support graft being slidable over an outer surface of the tubular vascular graft, wherein an outer diameter of the second mandrel and an outer diameter of the tubular vascular graft are sized such that the tubular support graft can slide over the outer surface of the tubular vascular graft when the segment of the vasculature is replaced.

In embodiments, the present disclosure further relates to an apparatus, comprising a tubular support graft comprising a 3-dimensional hydrogel matrix and cellular support structures, wherein the tubular support graft is configured to be concentrically positioned around a tubular vascular graft.

In embodiments, the present disclosure further relates to a method for replacing a segment of vasculature, comprising providing a tubular support graft concentrically arranged against an outer surface of a support mandrel, contacting at least a planar end of the support mandrel with at least a planar end of a vascular graft mandrel, and sliding the tubular support graft over an outer surface of a tubular vascular graft concentrically arranged against an outer surface of the vascular graft mandrel.

In embodiments, the present disclosure further relates to a method for forming a tubular support graft, comprising arranging a support mandrel within a tubular mold, a first end of the support mandrel and a first end of the tubular mold being fluidly sealed by a first end cap, injecting a mixture of hydrogel and cellular support structures within a volume formed between surfaces of the support mandrel and the tubular mold, fluidly sealing a second end of the support mandrel and a second end of the tubular mold via a second end cap, and removing the second end cap and the tubular sleeve after curing of the mixture to form the tubular support graft.

In embodiments, the present disclosure further relates to a method for forming a tubular support graft, comprising injecting a mixture of hydrogel and cellular support structures into a culture dish, after the mixture cures, wrapping the cured mixture around a support mandrel, and securing open ends of the wrapped cured mixture to form the tubular support graft.

In embodiments, the present disclosure further relates to a method for forming a tubular support graft, comprising rotating a support mandrel at a predetermined speed, depositing, via a 3-dimensional printer, droplets of a mixture of hydrogel and cellular support structures onto a surface of the rotating support mandrel, and after curing, removing the cured mixture from the rotating support mandrel to form the tubular support graft.

In embodiments, the present disclosure further relates to an apparatus, comprising a tubular support graft comprising a 3-dimensional hydrogel matrix and cellular support structures, wherein the tubular support graft is configured to be concentrically positioned around a vessel segment harvested from patient vasculature.

In embodiments, the present disclosure further relates to a method for augmenting a segment of patient vasculature, comprising harvesting the segment of patient vasculature, sliding the harvested segment of patient vasculature over a vascular graft mandrel such that the harvest segment is concentrically arranged against an outer surface of the vascular graft mandrel, providing a tubular support graft concentrically arranged against an outer surface of a support mandrel, contacting at least a planar end of the support mandrel with at least a planar end of the vascular graft mandrel, and sliding the tubular support graft over an outer surface of the harvested segment to augment the harvested segment.

In embodiments, the present disclosure further relates to a method for augmenting a segment of patient vasculature, comprising obtaining a vascular allograft, sliding the vascular allograft over a vascular graft mandrel such that the vascular allograft is concentrically arranged against an outer surface of the vascular graft mandrel, providing a tubular support graft concentrically arranged against an outer surface of a support mandrel, contacting at least a planar end of the support mandrel with at least a planar end of the vascular graft mandrel, and sliding the tubular support graft over an outer surface of the vascular allograft to augment the vascular allograft.

### BRIEF DESCRIPTION OF THE SEVERAL VIEWS OF THE DRAWINGS

FIG. 1 is a schematic illustration of the approach used to produce tissue-engineered vascular grafts, also referred to herein as tissue-engineered constructs. Each construct is generated in the laboratory by (step A) culturing human cells on a polymer scaffold that degrades as the cells produce extracellular matrix proteins to form (step B) a tissue. Cellular material is then removed, leaving (step C) an extracellular matrix construct, which may be refrigerated or stored at room temperature, or by some other storage means until the time of patient need. Cell-derived extracellular matrix protein constructs may be implanted without cells (step D, diameters ≥6 mm), or (step E) seeded with recipient endothelial cells for small diameter (3-4 mm) applications.

FIG. 2A is an illustration of a tubular mold (5) and a cylindrical mandrel (2). FIG. 2B is an illustration of a sleeve graft (1). The sleeve graft (1) can be produced by casting a hydrogel into the space between the cylindrical mandrel (2) and a tubular mold (5). As in FIG. 2B and the remaining Figures, the sleeve graft can be an islet hydrogel mixture comprising islets within the hydrogel. Upon curing/polymerization of the hydrogel, the sleeve graft and the mandrel can be removed from the mold.

FIG. 3A is an illustration of sleeve graft that can be produced by casting an islet and hydrogel mixture into a rectangular prism-shaped mold to create a flat sheet of islet-populated hydrogel FIG. 3B is an illustration demonstrating that the sheet can later be wrapped around a mandrel to form the sleeve graft by adhering the two ends via sutures, biocompatible glue, or polymer mesh.

FIG. 4 is an illustration of a sleeve graft produced by using 3D-printing. A fast-curing bio-ink containing islets can be injected onto a cylindrical mandrel to form the sleeve graft.

FIG. 5 is an illustration of components, including a human acellular vessel (HAV) in sterile solution within a closed silicone bag and an islet-populated sleeve graft in a separate closed container which keeps the sleeve graft sterile. The HAV and the sleeve graft can be delivered separately to the operation room, and they can be combined by sliding the sleeve graft onto the HAV by the surgeon.

FIG. 6A illustrates a step-wise process of combining an HAV and the sleeve graft, which may be islet-populated. At Step 1, a fibrin gel that is in the form of a tube (1) and populated with pancreatic islets is formed and placed onto a mandrel (2). At Step 2, an end of the mandrel (2) and an end of a second mandrel (4) carrying the HAV (3) are contacted. At Step 3, the islet-populated fibrin tube, or the islet-populated sleeve graft, can be slid over the HAV (3). Step 3 in FIG. 6A illustrates a configuration after the HAV (3) and the islet-populated sleeve graft (1) are combined. FIG. 6B is a flow diagram describing coupling of the sleeve graft and the tissue-engineered vascular graft. FIG. 6C is a rendering reflecting Step 3 of FIG. 6A, wherein an islet-populated sleeve graft is slide over an HAV (3).

FIG. 7A demonstrates a process of sliding the sleeve graft (1) onto the HAV (3) by the surgeon. FIG. 7B is an image of a hematoxylin and eosin-stained tissue section showing the sleeve graft layer (1) carrying islets around the HAV (3) before implantation (any detachment of the sleeve graft layer from the HAV was a caused by the tissue processing for histology).

FIG. 8A through FIG. 8D relate to performance of non-human primate (NHP) sized HAVs sheathed by islet-populated sleeve graft. In particular, as shown in FIG. 8A, the sheathed HAVs were cultured in a bioreactor mimicking in vivo conditions, where the oxygen level outside the sheathed HAVs was low (i.e., 40 mmHg O₂) while the lumen of the sheathed HAVs remained well oxygenated (i.e., >95 mmHg O₂). The sheathed HAVs were cultured in an incubator for 6 days, the last 2 days being under hypoxic conditions. Cell viability of islet cells was evaluated using propidium iodide, a dye that is permeant to comprised cell membranes. After 6 days of culture, 92% of free islets in normoxia were alive while, as shown in FIG. 8B, the viability of islets within the sheath and islets in hypoxia were 80% and 28%, respectively. FIG. 8C is a graphical representation of cell viability under variable conditions. As shown, free islets in hypoxia and islets in fibrin in hypoxia experienced decreased cell viability compared to islets within the sleeve grafts. The islet density in the sleeve grafts were 125K islet equivalent (IEQ) per 40 cm and 250K IEQ per 40 cm. FIG. 8D is a graphical representation of insulin secretion by free islets and islet-populated sleeve grafts in response to variable glucose levels. As shown, insulin secretion by islet-populated sleeve grafts is comparable to insulin secretion by free islets at normoxia. These findings show that when the islet-populated sheath is placed outside of a vascular graft, the oxygen-rich flow in the lumen of the vascular graft supports the viability of the islet cells within the sleeve graft though the oxygen level in the surrounding environment is low.

### DETAILED DESCRIPTION

### Definitions

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, a “tissue-engineered construct” refers to a three-dimensional structure produced primarily by growth in vitro using living mammalian tissue or cells. The construct may include one or more types of tissue, and each tissue may include one or more types of cells. A tissue-engineered construct is distinguished from an explant of a corresponding natural tissue in that the growth of the construct occurs in vitro.

As used herein, a “porous substrate” refers to a three-dimensional substrate of a biocompatible material which is suitable for attachment or adherence of mammalian cells, and which is sufficiently porous to allow for the infiltration of seeded cells, and the diffusion of nutrients and waste products to and from cells adhered to the substrate, including cells adhered within the interior pores or interstitial spaces of the substrate. Thus, a porous substrate has pores or interstitial spaces interspersed through its structure, and in fluid communication with the exterior, such that cells may infiltrate into the interior of the substrate. The pores or interstitial spaces may be roughly spheroidal spaces, such as the pores in a sponge-like material, or may be longitudinally extended and intersecting spaces, such as the inter-fiber spaces in a fibrous mesh material or may be of any other arbitrary shape. As used herein, no distinction is made between the “pores” of sponge-like materials, the “interstitial spaces” of fibrous mesh materials, or the arbitrarily shaped “spaces” of any other materials, and the term “porous” embraces materials characterized by any of these.

As used herein, the term “synthetic polymer” refers to a non-naturally occurring polymer made by, for example, ex vivo synthesis, and physically distinguishable from naturally occurring polymers. Thus, the term is used herein merely to distinguish synthetic polymers, such as those described and enabled herein, from such naturally-occurring polymers as collagen, elastin, polysaccharides, cellulose, chitosan, and the like. A synthetic polymer may include one or more naturally-occurring subunits, such as naturally occurring amino acids or saccharide units, in an otherwise non-natural polymer (e.g., copolymers of lysine or arginine with lactic acid or glycolic acid).

As used herein, the term “proteinaceous polymer” means a polymer consisting essentially of naturally-occurring or chemically modified amino acids residues joined by peptide linkages. Proteinaceous polymers of the invention may be naturally-occurring polymers which are extracted from animal tissues (e.g., collagen obtained from connective tissues), may be recombinantly produced polymers obtained from genetically engineered organisms (e.g., bacteria engineered to produce elastin), or may be produced in vitro by chemical synthesis. Thus, for example, as used herein, the term embraces such naturally-occurring proteinaceous polymers as collagen, elastin, fibronectin, laminin and the like. A proteinaceous polymer may also include one or more non-naturally-occurring subunits, such as modified amino acids (e.g., acylated, sulfonated, glycosylated, or otherwise conjugated through reactive amino acid side chain groups to moieties which increase hydrophilicity or provide better cell-adhesion characteristics), or may include non-peptide linkages joining two or more proteinaceous fragments (e.g., polypeptides or modified polypeptides copolymerized with polyesters, polyanhydrides).

As used herein, the term “cellular support structures” refers to any component, excepting the material and structure of the tubular support graft, itself, that supports cellular viability and graft viability in vivo. In embodiments, the cellular support structures refer to cells, cellular matter, and/or proteins. The cellular support structures may include, as will be described below, drugs, drug-releasing particles, other cell types, organoids, therapeutic agents, growth factors, enzymes, peptides, nucleic acids, molecules, and the like.

As used herein, the term “organoid” refers to a miniaturized, self-organized three-dimensional tissue culture derived from cells and, particularly, stem cells. Such cultures can be crafted to replicate much of the complexity of an organ, or to express selected aspects of it like producing only certain types of cells. Organoids can range in size from less than the width of a human hair to around five millimeters, depending on experimental constraints. Additionally, organoids can be derived from one or a few cells from a tissue, adult stem cells, pluripotent stem cells, hematopoietic stem cells, embryonic stem cells, or induced pluripotent stem cells, which can self-organize in three-dimensional culture owing to their self-renewal and differentiation capacities.

The present disclosure describes a system, apparatus, and methods for replacing a vascular segment or organ segment within host vasculature. The system, for instance, includes a tubular support graft arranged concentrically against an outer surface of a tubular vascular graft. In embodiments, the tubular vascular graft may be native tissue harvested from patient vasculature or may be an allograft. The native tissue or allograft may be cellularized or decellularized. In embodiments, the tubular vascular graft may be an engineered vascular graft and may be cellularized or acellular. The engineered vascular graft may be referred to herein interchangeably as a tissue-engineered construct. In embodiments, the tubular support graft can be laden with organ pieces, tissue pieces, cells, and the like that secrete therapeutic agents, or can be laden with the therapeutic agents, themselves. In instances where the tubular support graft is laden with either therapeutic agent-generating components (e.g., organ pieces, tissue pieces, cells) or therapeutic agents, themselves, the tubular support graft may be referred to berein as a doped tubular support graft.

In embodiments where a tissue-engineered construct is, at implantation, cell-laden with e.g., endothelial cells, smooth muscle cells, and the like, a doped tubular support graft improves survival and functionality of cells in the tissue-engineered construct.

In embodiments where a tissue-engineered construct is acellular upon implantation, a doped tubular support graft can prevent thrombosis and stenosis and can facilitate (1) cellular infiltration into the tissue-engineered construct and (2) viability of the implanted tissue-engineered construct, at large.

In embodiments, the tubular support graft comprises cellular support structures.

In embodiments, the tubular support graft comprises fibrin gel (formed from fibrinogen and thrombin), collagen, agarose, silk, chitosan, alginate, gelatin methacrylovl, elastin, and the like, or any combinations thereof.

In embodiments, the tubular support graft comprises, as structural reinforcement, poly(glycolic acid) (PGA), poly(lactic acid) (PLA), poly(lactic-co-glycolic acid) (PLGA), polyethylene terephthalate (PET), polycaprolactone (PCL), poly(lactide-co-caprolactone) (PLCL), poly(methyl methacrylate) (PMMA), poly(vinyl alcohol) (PVA), poly(ethylene glycol) (PEG) polytetrafluoroethylene (PTFE), expanded polytetrafluoroethylene (ePTFE), polyurethane (PU), poly(glycerol-co-sebacate) (PGS), and poly(ethylene glycol) diacry-late (PEGDA), and the like, or any combinations thereof.

In embodiments, the tubular vascular graft and/or the tubular support graft are formed by electrospinning, 3-dimension printing or bioprinting.

In embodiments, the doped tubular support graft can include drugs, drug-releasing microparticles, and/or drug-releasing nanoparticles. In embodiments, the doped tubular support graft can include cells and/or organoids capable of producing therapeutic agents including proteins such as cytokines, enzymes, and the like. For instance, the doped tubular support graft can include endothelial cells. In embodiments where the doped tubular support graft is cellularized, the cells may be stem-cell derived, genetically modified, and/or may have a low immunogenic profile.

In an embodiment, the tubular support graft comprises pancreatic islet cells which may form pancreatic islets. In an example, the pancreatic islet-comprising tubular support graft produces insulin in response to glucose and the produced insulin diffuses and/or transits through the tubular vascular graft and into patient vasculature.

In embodiments, the doped tubular support graft can include proteins (e.g., cytokines, enzymes, and the like) directly incorporated within the tubular support graft.

In embodiments, the doped tubular support graft can include growth factors such as vascular endothelial growth factor (VEGF), hepatocyte growth factor (HGF), fibroblast growth factor (FGF), transforming growth factor beta 1 (TGFβ1), and platelet derived growth factor (PDGF), or any combination thereof to facilitate vascularization of the tubular support graft following implantation.

In embodiments where a tissue-engineered construct is cell-laden at implantation, the doped tubular support graft can include molecules such as Fas ligands, nitric oxide donors, or any combination thereof to target activated T-cells that may attack the tubular support graft or the cellularized tissue-engineered construct.

As it particularly relates to engineered vascular grafts as the tubular vascular grafts of the present disclosure, which may also be referred to as tissue-engineered vascular grafts (TEVGs), different techniques can be used for TEVG production, including electrospinning, decellularization, lyophilization, biotubes, and 3-dimensional (3D) bioprinting, among others. Regarding electrospinning, electrospinning produces porous and fibrous scaffolds from polymers, thereby enhancing the transfer of nutrients and residues through the scaffold. The alignment of the nanofibers allows the scaffold's strength to be increased to promote cell alignment. Regarding decellularization, TEVGs can be decellularized to obtain extracellular matrices. These matrices mimic the biological properties of native blood vessels. To this end, the TEVGs contain functional proteins capable of promoting cell recruitment. Regarding lyophilization, lyophilization is a physical dehydration and freezing technique that reduces calcification and provides a stable graft. Regarding 3D bioprinting, 3D printing has been used to manufacture TEVGs and provides an adequate cell distribution with a high cell density.

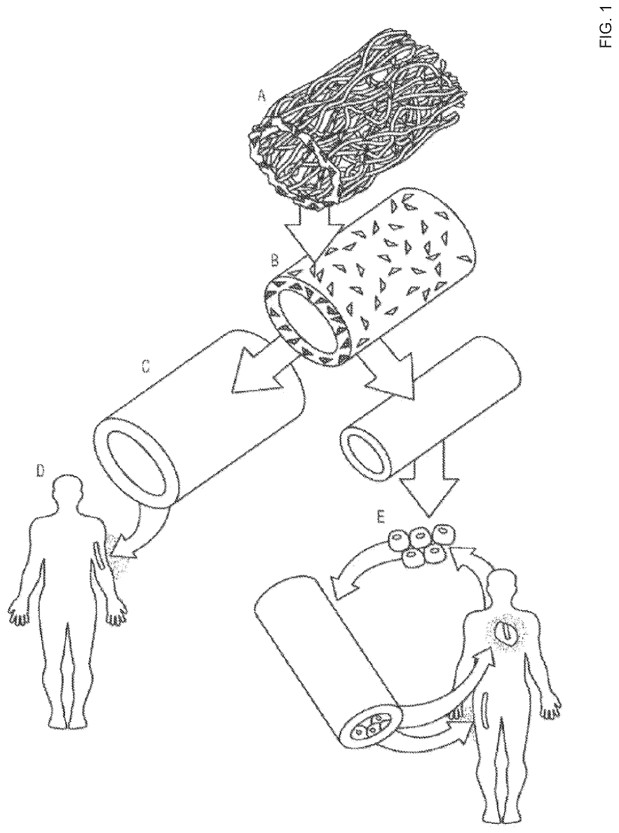

Referring now to the Drawings. FIG. 1 is a schematic illustration of an approach used in the present disclosure is described. While FIG. 1 describes a tissue engineered construct that is formed, decellularized and, optionally, re-seeded with cells, it should be appreciated that the same protocol from step C or step E may be followed if the tubular vascular graft is a native vessel harvested from patient vasculature. In fact, when the tubular vascular graft is a native vessel harvested from patient vasculature, decellularization may or may not be performed. Similarly, when the tubular vascular graft is an allograft, the allograft may be used within the protocol of FIG. 1 from either of step C or step E.

In embodiments, and to produce engineered tubular vascular grafts, also referred to herein as tissue-engineered constructs, each tissue-engineered construct is first generated in the laboratory by (step A) culturing human cells on a scaffold that degrades as the cells produce extracellular matrix proteins to form (step B) a tissue. Cellular material is then removed by decellularization protocols, and an extracellular matrix construct remains at step C. The decellularized conduit may then be refrigerated or stored at room temperature, or by some other storage means until the time of patient need. These cell-derived extracellular matrix protein constructs may be implanted without cells (step D, diameters ≥6 mm), or (step E) seeded with recipient cells, such as endothelial cells for e.g., small diameter (3-4 mm) applications.

In embodiments, the scaffold of the tubular vascular graft may be a synthetic polymer such as PLA, PGA, PLGA, PLCL, and/or PCL.

In embodiments, steps A-C are performed regardless of whether the implanted tubular vascular graft is to be acellular or cellular. The cells seeded at step A may be allogeneic, autologous, syngeneic, or xenogeneic. The cells can be seeded onto the scaffold at about 0.5×10⁶ cells per cm length of tissue-engineered construct to about 2×10⁶ cells per cm length of tissue-engineered construct. Typically, after step B, cells used in making the conduits are killed and/or removed prior to use. In other words, the tubular vascular graft is decellularized. The killing and/or removal of cells diminishes the potential for adverse 35 immune reactions. Killing and/or removal of cells leaves less than 50%, less than 75%, less than 80%, less than 85%, less than 90%, or less than 95% of the cells viable, as assessed by trypan blue staining, nucleotide incorporation, or protein synthesis. Remaining extracellular matrix is 40 highly conserved among individuals, and among species, rendering it less likely to provoke an adverse immune reaction than live cells.

In embodiments, vascular smooth muscle cells are used to make the extracellular matrix at step B. These can be isolated from any vasculature of a human or other mammal, including from the aorta. Much of the secreted extracellular matrix comprises collagen Collagen may comprise at least 5%, at least 10%, at least 15%, at least 20%, at least 25%, at least 30% of the extracellular matrix. Typically, the extracellular matrix is grown until it achieves a thickness of at least 50 microns, at least 100 microns, at least 150 microns, at least 200 microns, at least 250 microns, at least 300 microns, at least 400 microns, or at least 500 microns. Diameter of the conduits may be controlled during manufacturing. Typically, these may have an internal diameter of at least 10, at least 15, at least 20, at least 25, at least 30, at least 35, at least 40, at least 45, or at least 50 mm.

In embodiments, wherein the tissue-engineered construct is again seeded with the patient's cells prior to implantation at step E, cell seeding facilitates cell fixation and infiltration, thus improving graft endothelialization. The cells may be allogeneic or autologous. The cells may include vascular cells such as endothelial cells, fibroblast, and endothelial cells. The cells may be mesenchymal stem cells (MSCs) obtained from different origins, such as adipose tissue, bone marrow, and umbilical vein blood. MSCs are a promising cell type for TEVG because improve the patency due to their anti-thrombogenic property, and they can also recruit endothelial cells on site. A wide variety of vascular cells could be obtained with induced pluripotent stem cells (iPSCs) because they could be induced into specific lineages, such as smooth muscle cells or endothelial cells. The cells may include bone marrow-derived mononuclear cells, such as MSCs, immune-related cells, and hematopoietic stem cells, which exhibit an anti-thrombotic effect. Although various cell types can be used, endothelial cells are the most commonly used cell in the design of TEVGs, as they provide anticoagulant effects and improve endothelialization.

In view of the above, the present disclosure describes a tubular support graft, also referred to herein as a sleeve, that can be attached as a separate tissue layer onto an outer surface of a blood vessel, such as TEVG, a harvested native vessel segment, or an allograft, and immediately provide physiologic support within the host vasculature. For instance, the tubular support graft may comprise pancreatic islets that produce insulin in response to glucose.

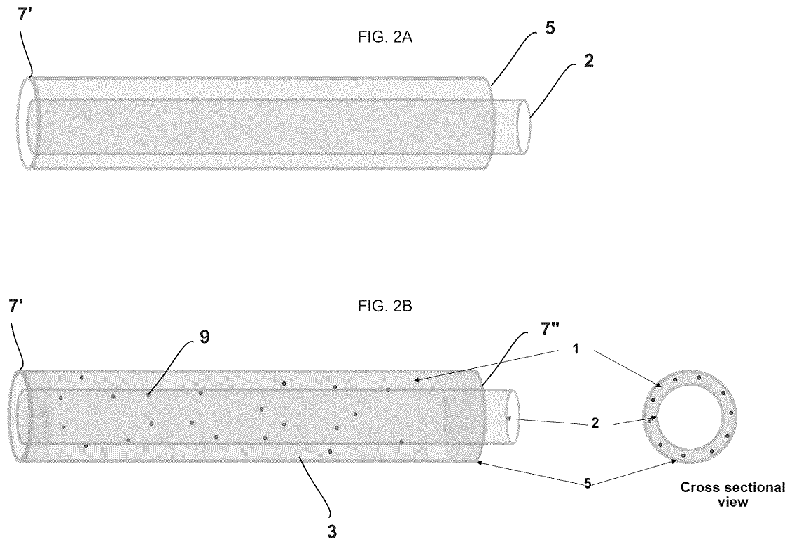

FIGS. 2-4 provide illustrations of such a tubular support graft and methods for manufacturing the same.

The tubular support graft may be formed from fibrin gel (formed from fibrinogen and thrombin), collagen, agarose, silk, chitosan, alginate, gelatin methacryloyl, elastin, and the like, or any combinations thereof. Each tubular support graft can be defined by a length, an inner diameter, and an outer diameter, the distance between the inner diameter and the outer diameter being referred to as a depth or a thickness.

In an embodiment, the length of the tubular support graft can be shorter than, longer than, or about the same length as a corresponding tubular vascular graft. In embodiments, the length of the tubular support graft can be between about 1 cm and about 100 cm. For instance, the length of the tubular support graft can be about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 25 cm, about 30 cm, about 35 cm, about 40 cm, about 45 cm, about 50 cm, about 60 cm, about 70 cm, about 80 cm, about 90 cm, or about 100 cm. In an embodiment, the length of the tubular support graft can be about 10 cm to about 40 cm.

In an embodiment, the inner diameter of the tubular support graft can be greater than about 1 mm. In embodiments, the inner diameter of the tubular support graft can be about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 25 mm, about 30 mm, about 35 mm, about 40 mm, about 45 mm, or about 50 mm. In an embodiment, the inner diameter of the tubular support graft is about 3 mm to about 20 mm.

In an embodiment, the outer diameter of the tubular support graft can be greater than about 1 mm. In embodiments, the outer diameter of the tubular support graft can be about 1mm, about 2 mm, about 3 mm, about 4 mm, about 5 mm, about 6 mm, about 7 mm, about 8mm, about 9 mm, about 10 mm, about 11 mm, about 12 mm, about 13 mm, about 14 mm, about 15 mm, about 16 mm, about 17 mm, about 18 mm, about 19 mm, about 20 mm, about 25 mm, about 30 mm, about 35 mm, about 40 mm, about 45 mm, or about 50 mm. In an embodiment, the outer diameter of the tubular support graft is about 3 mm to about 20 mm.

In an embodiment, the inner diameter and the outer diameter are sized according to requirements of an intended use. For instance, when the diameter of a segment of vasculature to be replaced is 3 mm, the inner diameter of the tubular vascular graft must be sized, accordingly, and the inner diameter of the tubular support graft must be sized based on an outer diameter of the tubular vascular graft. Moreover, the outer diameter of the tubular support graft must then be sized to provide a particular thickness (i.e., wall thickness) of the tubular support graft. In embodiments, this thickness is about 0.3 mm to about 1.5 mm, wherein the thickness is approximately uniform along a circumference and a length of the tubular support graft. In an embodiment, the thickness of the tubular support graft can be about 0.4 mm to about 0.6 mm, wherein the thickness is approximately uniform along a circumference and a length of the tubular support graft.

In embodiments, the tubular support graft comprises, as structural reinforcement. poly(glycolic acid) (PGA), poly(lactic acid) (PLA), poly(lactic-co-glycolic acid) (PLGA), polyethylene terephthalate (PET), polycaprolactone (PCL), poly(lactide-co-caprolactone) (PLCL), poly(methyl methacrylate) (PMMA), poly(vinyl alcohol) (PVA), poly(ethylene glycol) (PEG) polytetrafluoroethylene (PTFE), expanded polytetrafluoroethylene (ePTFE), polyurethane (PU), poly(glycerol-co-sebacate) (PGS), and poly(ethylene glycol) diacry-late (PEGDA), and the like, or any combinations thereof.

In embodiments, the tubular support graft can be doped with cellular support structures. In embodiments, the doped tubular support graft can include, as cellular support structures, drugs, drug-releasing particles (i.e., microparticles, nanoparticles), and cells and/or organoids capable of producing therapeutic agents including proteins such as cytokines, enzymes, and the like. For instance, the doped tubular support graft can include pancreatic islet cells and/or endothelial cells. In embodiments where the doped tubular support graft is cellularized, the cells may be stem-cell derived, genetically modified, and/or may have a low immunogenic profile. In embodiments, the cellular support structures within the doped tubular support graft can include iPSC-derived pancreatic islet cells. The iPSC-derived pancreatic islet cells can include β cells, a cells, F cells, and/or δ cells.

In embodiments, the doped tubular support graft can include, as cellular support structures, proteins. In embodiments, the proteins can include cytokines, enzymes, and the like directly incorporated within the tubular support graft, as well as growth factors such as VEGF, HGF, FGF, TGFβ1, and PDGF, or any combination thereof to facilitate vascularization of the support graft following implantation.

In embodiments where a tissue-engineered construct is cell-laden at implantation, the doped tubular support graft can include, as cellular support structures, molecules such as Fas ligands, nitric oxide donors, or any combination thereof to target activated T-cells that may attack the tubular support graft or the cellularized tissue-engineered construct. Fas ligands are a type-II transmembrane protein that belong to the tumor necrosis family. The binding of Fas ligands with its receptor induces apoptosis. Nitric oxide is involved in the suppression of T-cell proliferation.

In embodiments, the above-described tubular support graft (and/or the tubular vascular graft) can be formed by electrospinning, 3-dimension printing or bioprinting, casting, molding, planar cell culture, folding/suturing, and the like.

As shown in FIG. 2A and FIG. 2B, an assembly comprising a tubular support graft 1 may be formed by casting between a support mandrel 2 and a tubular mold 5. As in FIG. 2A, the tubular mold 5 may have an inner diameter larger than an outer diameter of the support mandrel 2 to ensure the polymerized tubular support graft 1 will have a desired thickness. As in FIG. 2B, a mixture of a hydrogel 3 and cellular support structures 9, if present, can be injected into the space between the tubular mold 5 and the support mandrel 2. To ensure the injected mixture is maintained between surfaces of the tubular mold 5 and the support mandrel 2, a first end cap 7′ may be added at a first end of the assembly. After injecting the mixture, a second end cap 7″ may be added at a second end of the assembly to ensure the injected mixture is maintained between the surfaces of the tubular mold 5 and the support mandrel 2. After injecting the mixture and securing the ends of the assembly, polymerization of the tubular support graft 1 can proceed. The cross-sectional view of FIG. 2B illustrates a thickness of the tubular support graft 1 with cellular support structures 9 embedded within a hydrogel 3. Once polymerized, the tubular support graft 1 can be optionally removed and stored until use.

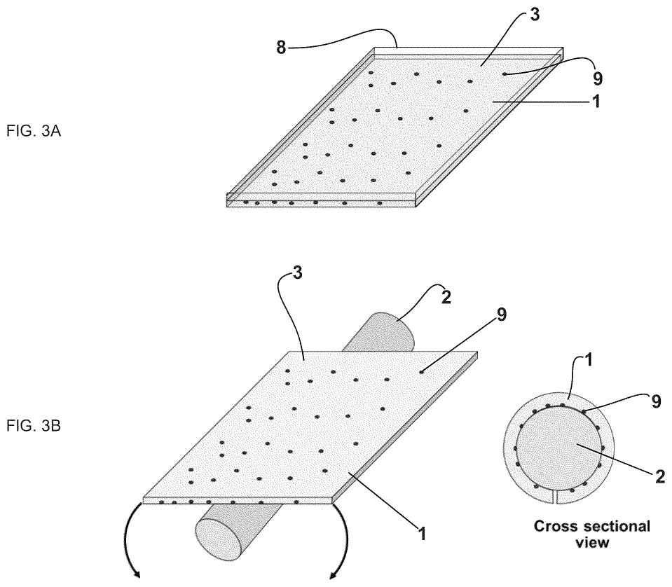

As shown in FIG. 3A and FIG. 3B, a tubular support graft 1 may be formed by, first, casting a mixture of hydrogel 3 and cellular support structures 9 onto a planar surface of a culture dish 8, as in FIG. 3A. A volume of the mixture cast into the culture dish 8 may be such that, once cured, the tubular support graft 1 has a desired thickness. Second, after the mixture of the hydrogel 3 and the cellular support structures 9 cures, as in FIG. 3B, the tubular support graft 1 may be wrapped around a support mandrel 2. In embodiments, the ends of the tubular support graft 1 may be connected by a biological adhesive, by sutures, by polymer mesh, or by another fixation means. As shown in FIG. 3B, the cellular support structures 9 may be preferentially arranged toward an inner surface of the tubular support graft 1, in certain embodiments. The cross-sectional view of FIG. 3B illustrates a thickness of the tubular support graft 1 with cellular support structures 9 embedded within a hydrogel 3. After fixing the ends of the tubular support graft 1, the tubular support graft 1 can be optionally removed and stored until use.

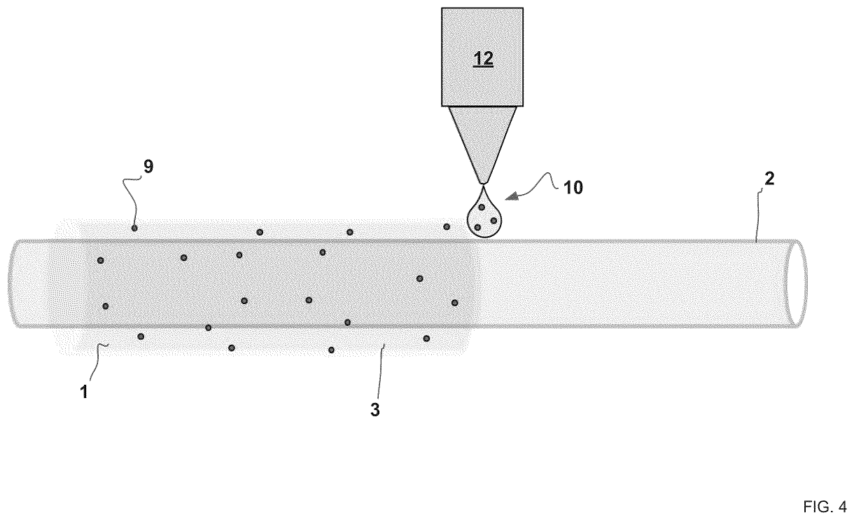

As shown in FIG. 4, a tubular support graft 1 may be formed by 3D printing or bioprinting. A droplet 10 of a mixture of hydrogel 3 and cellular support structures 9 may be dropped onto a support mandrel 2 from a 3D bioprinter 12. The support mandrel 9 may be rotated at a predetermined speed to ensure that the droplet 10 is dispersed along the surface of the support mandrel 9 and results in a predetermined thickness of the tubular support graft 1. To this end, the 3D bioprinter 12 includes a head that translates along a length of the support mandrel 2 based on a desired length of the tubular support graft 1. Once polymerized, the tubular support graft 1 can be optionally removed from the support mandrel 2 and stored until use.

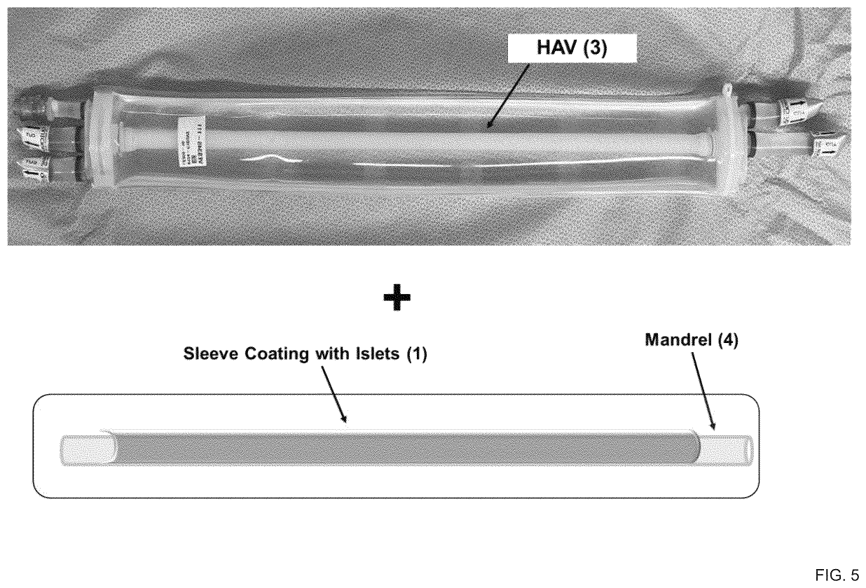

FIG. 5 is an illustration of a tubular vascular graft and a tubular support graft prior to assembly. A closed silicone bag can contain a tissue-engineered construct, or HAV 3, in a sterile solution. A tubular support graft, or sleeve comprising islets 1 (i.e., sleeve coating with islets), can be maintained in a separate closed container, concentrically arranged against a surface of a mandrel 4, which keeps the tubular support graft 1 sterile. The HAV 3 and the tubular support graft 1 can be delivered separately to the operating room, where the surgeon can combine the tubular support graft 1 and the HAV 3 by sliding the tubular support graft 1 onto the HAV 3.

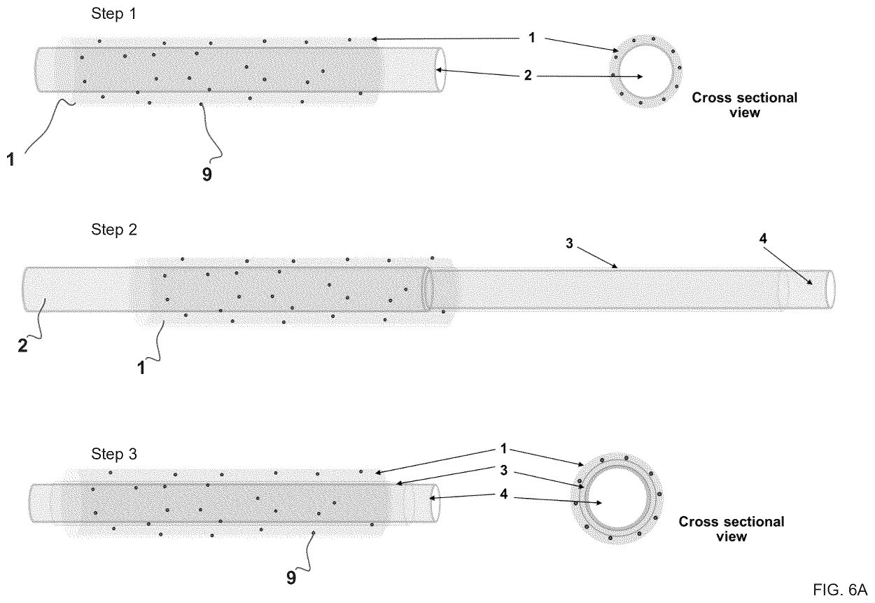

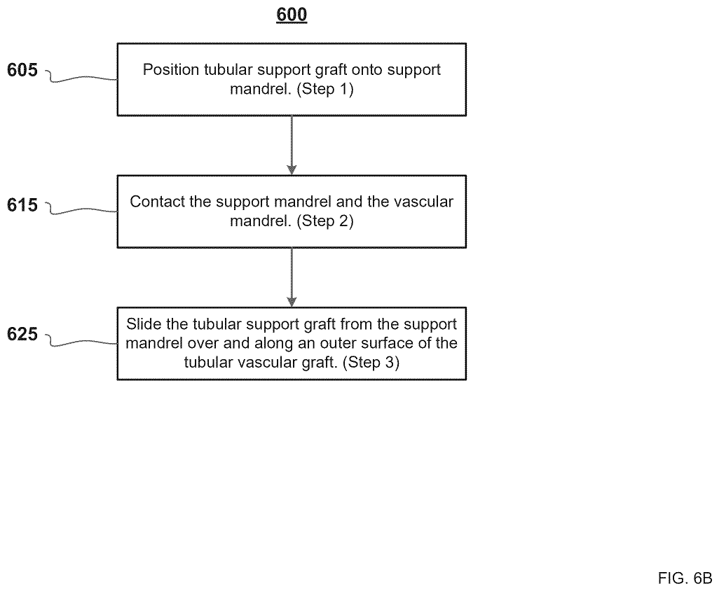

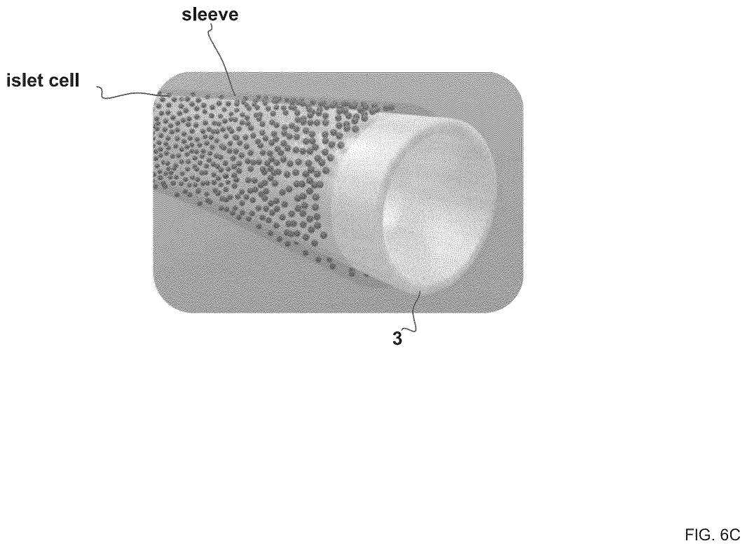

Regarding clinical use, FIG. 6A and FIG. 6B illustrate and describe a process for assembling a tubular vascular graft and a tubular support graft in the operating room. Description of Step 1 through Step 3 will be made in view of the illustrations of FIG. 6A and the flow diagram of FIG. 6B.

In method 600 of FIG. 6B, cellular support structures 9 may comprise pancreatic islets, the tubular support graft 1 may comprise fibrin gel, and the tubular vascular graft may be a decellularized TEVG.

At step 605, or Step 1, the tubular support graft 1 may be positioned onto a final support mandrel 2. The final support mandrel 2 can be larger than the support mandrel used during initial fabrication of the tubular support graft 1. The final support mandrel 2 may be configured to have an outer diameter relatively larger than an outer diameter of the vascular mandrel 4. In fact, the final support mandrel 2 may be configured to have an outer diameter relative larger than an outer diameter of the tubular vascular graft 3 concentrically arranged against a surface of the vascular mandrel 4. Such a geometric relationship allows for efficiency in concentrically arranging the tubular support graft 1 over the tubular vascular graft 3.

At step 610, or Step 2, ends of the final support mandrel 2 and the vascular mandrel 4 can be contacted or opposed. In certain cases, the vascular mandrel 4 can be inserted at least partially into a lumen of the final support mandrel 2. At step 615, the tubular support graft 1 can be slid off the final support mandrel 2 and onto the tubular vascular graft 3 and across an outer surface of the tubular vascular graft 3.

As shown in Step 3 of FIG. 6A, the assembled system includes the tubular support graft 1 concentrically arranged against the outer surface of the tubular vascular graft 3 disposed on the vascular mandrel 4. The assembled system can be appreciated further in view of the cross-sectional view.

### EXAMPLES

### Example 1

A glass tubular mold with an inner diameter of 8 mm was capped from one end by using a silicone plug and filled with sterile distilled water with 0.5% Pluronic F127. Pluronic treatment was applied for 30 minutes and then the glass tubular mold was briefly rinsed with distilled water. A mandrel rod with 7 mm outer diameter was inserted into the glass tubular mold (shown in FIG. 2). A piece of silicone tubing was used between the glass tubular mold and the mandrel rod to seal one end of the glass tubular mold to center the mandrel mold inside the glass tubular mold.

Fibrinogen powder from human plasma was dissolved in phosphate buffered saline (PBS) or culture medium at 37° C. for 1-2 hours until a transparent solution with a desired fibrinogen concentration of ≈40.9 mg/mL was obtained. Then, the fibrinogen solution was filter sterilized in a biosafety cabinet. Sterile calcium chloride (CaCl) solution was added to the fibrinogen solution and the mixture was placed on ice. Thrombin solution from human plasma was placed on ice in a separate conical tube.

Pancreatic islets (i.e., cellular support structure) were collected from the culture flask and centrifuged at 200 rcf for 3 minutes The supernatant was discarded, and the islet pellet was gently resuspended in fibrinogen solution on ice. Thrombin solution was added to the fibrinogen-CaCl-islet mixture and mixed well by pipetting the fibrinogen solution up and down on ice. The islet/fibrinogen/CaCl/thrombin mixture was pipetted into the space between the glass tubular mold and the mandrel rod (shown in FIG. 2). The second end of the glass tubular mold was sealed, and the mandrel rod was centered completely with a second segment of silicone tubing. The assembly and its contents were placed into a sterile closed tray and incubated at 37° C. for 15-20 minutes to allow the polymerization of the fibrin gel. After gelation, the islet-populated fibrin sleeve (i.e., tubular support graft) was removed from the assembly by pulling the mandrel rod out from the glass tubular mold. The islet-populated fibrin sleeve was first transferred to a new mandrel rod with an outer diameter of 8 mm by gently sliding it from the original 7-mm mandrel rod to the 8-mm mandrel rod. Transferring the islet-populated fibrin sleeve to a larger mandrel rod allows for easier sliding onto a tubular vascular graft, which has an approximate outer diameter of 7-mm. The 8-mm mandrel rod carrying the islet-populated fibrin sleeve was placed in a cylindrical bottle filled with culture medium and stored at 4° C. until implantation.

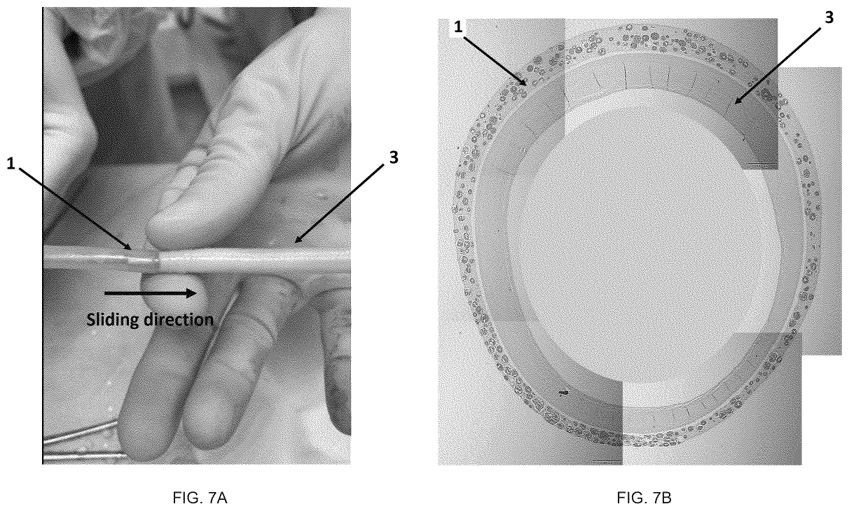

For the implantation, a tubular vascular graft was removed from its original container, and a mandrel rod with a 5-mm outer diameter was inserted into the tubular vascular graft. The tubular vascular graft was a decellularized TEVG. Next, the 8-mm mandrel rod carrying the islet-populated fibrin sleeve, or tubular support graft, was taken from its container and the tip of the 5-mm mandrel rod carrying the tubular vascular graft was inserted into the 8-mm mandrel rod carrying the tubular support graft. Then, the tubular support graft was transferred from the 8-mm mandrel to the tubular vascular graft, as an outer jacket, by gently sliding the tubular support graft onto the tubular vascular graft (as shown in FIG. 7A and FIG. 7B). Finally, the 5-mm mandrel rod is removed from the tubular vascular graft and the assembled system is ready to proceed to implantation.

Shown in FIG. 7A, a surgeon slides a tubular support graft 1 off a support mandrel and onto a tubular vascular graft 3 and across an outer surface of the tubular vascular graft 3. FIG. 7B is an image of a hematoxylin and eosin-stained tissue section confirming the concentric arrangement of the tubular support graft 1 and the tubular vascular graft 3. Any observable detachment of the tubular support graft from the tubular vascular graft was caused by tissue processing for histology.

### Example 2

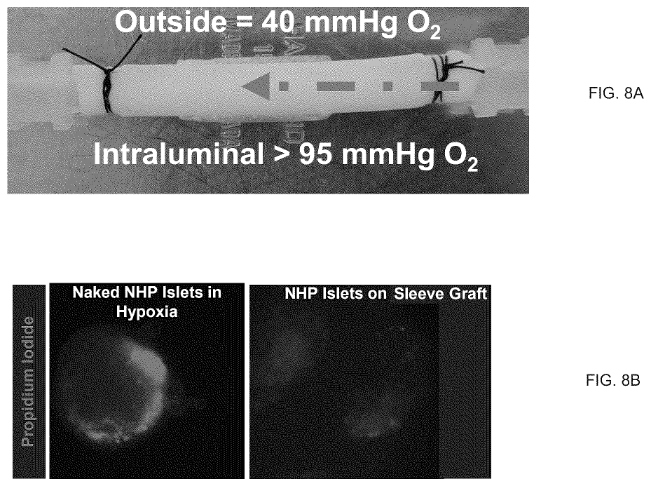

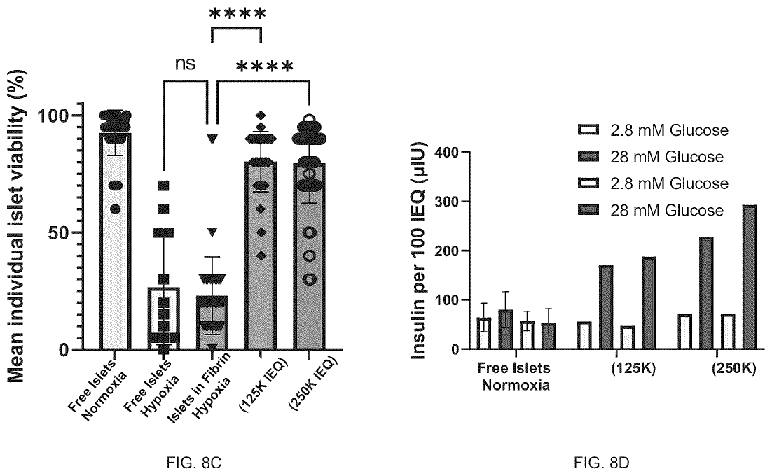

FIG. 8A through FIG. 8D relate to performance of non-human primate (NHP) sized HAVs sheathed by islet-populated sleeve grafts. In particular, as shown in FIG. 8A, the sheathed HAVs were cultured in a bioreactor mimicking in vivo conditions, where the oxygen level outside the sheathed HAVs was low (i.e., 40 mmHg O₂) while the lumen of the sheathed HAVs remained well oxygenated (i.e., >95 mmHg O₂: “normoxia”). The sheathed HAVs were cultured in the incubator for 6 days.

Cell viability of islet cells was evaluated using propidium iodide, a dye that is permeant to comprised cell membranes. After 6 days of culture, 92% of free islets in normoxia were alive while, as shown in FIG. 8B, the viability of islets within the islet-populated sleeve graft and islets in hypoxia were 80% and 28%, respectively. FIG. 8C is a graphical representation of cell viability under variable conditions. As shown, free islets in hypoxia and islets in fibrin in hypoxia experienced decreased cell viability compared to islets within the sleeve grafts. The islet density in the sleeve grafts were 125K islet equivalent (IEQ) per 40 cm and 250K IEQ per 40 cm. FIG. 8D is a graphical representation of insulin secretion by sleeve grafts in response to variable glucose levels. As shown, insulin secretion by free islets and islet-populated sleeve grafts is comparable to insulin secretion by free islets at normoxia.

There have been many attempts to implant cellular tissue-engineered structures (e.g., tissue-engineered liver, pancreas, kidney etc.), but these attempts have failed. For instance, these attempts suffer from a lack of incorporation of these cells into the patient's blood stream. Therefore, as in the present disclosure, depositing desired cells, organ pieces, and the like around a vessel, such as an HAV, and within a tubular support graft is an effective way of maintaining viable and functional cells, as the “implanted” cells are in close proximity to the blood supply on the outer surface of the HAV. Further, these attempts are hampered by the time intensive and technically demanding process of coating a cellular layer onto a vascular graft in the operating room. Moreover, coating a tissue-engineered vascular graft before shipping to the operating room would require removing the graft from its original packaging, which would compromise its sterility and all approved and/or patented features of the packaging. Therefore, the present disclosure allows for the sterile delivery of the tubular support graft to the operating room.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE INVENTION

Notwithstanding the appended claims, the disclosure sets forth the following numbered embodiments:
(1) A system for replacement of a segment of vasculature, comprising a tubular vascular graft concentrically arranged against an outer surface of a first mandrel, and a tubular support graft concentrically arranged against an outer surface of a second mandrel, the tubular support graft being slidable over an outer surface of the tubular vascular graft, wherein an outer diameter of the second mandrel and an outer diameter of the tubular vascular graft are sized such that the tubular support graft can slide over the outer surface of the tubular vascular graft when the segment of the vasculature is replaced.
(2) The system of (1), wherein the tubular vascular graft is a construct comprising a 3-dimensional proteinaceous extracellular matrix.
(3) The system of either (1) or (2), wherein the 3-dimensional proteinaceous extracellular matrix has been subjected to decellularization, and wherein the construct was formed by seeding a substrate with cells and maintaining the cells under conditions suitable for growth of the cells, whereby the proteinaceous extracellular matrix is formed around the cells.
(4) The system of any one of (1) to (3), wherein the substrate is polyglycolic acid.
(5) The system of any one of (1) to (4), wherein the proteinaceous extracellular matrix comprises collagen.
(6) The system of any one of (1) to (5), wherein the tubular vascular graft comprises smooth muscle cells.
(7) The system of any one of (1) to (6), wherein the tubular vascular graft comprises endothelial cells.
(8) The system of any one of (1) to (7), wherein the tubular vascular graft comprises smooth muscle cells toward the outer surface of the tubular vascular graft and endothelial cells toward an inner surface of the tubular vascular graft.
(9) The system of any one of (1) to (8), wherein the tubular vascular graft comprises autologous cells.
(10) The system of any one of (1) to (9), wherein the tubular support graft is a construct comprising a 3-dimensional hydrogel matrix and cellular support structures.
(11) The system of any one of (1) to (10), wherein the cellular support structures include at least one of a drug, a drug-releasing microparticle, and a drug-releasing nanoparticle.
(12) The system of any one of (1) to (11), wherein the cellular support structures include at least one of a cell, an organoid, and a tissue.
(13) The system of any one of (1) to (12), wherein the cell is a pancreatic islet cell.
(14) The system of any one of (1) to (13), wherein the tubular support graft further comprises pancreatic islets.
(15) The system of any one of (1) to (14), wherein the cellular support structures include at least one protein.
(16) The system of any one of (1) to (15), wherein the cellular support structures include at least one of a vascular endothelial growth factor, a fibroblastic growth factor, a platelet derived growth factor, and a hepatocyte growth factor.
(17) The system of any one of (1) to (16), wherein the cellular support structures include at least one of a Fas ligand, and a nitric oxide donor.
(18) The system of any one of (1) to (17), wherein the cellular support structures include endothelial cells.
(19) The system of any one of (1) to (18), wherein the tubular support graft is formed by one of molding, casting, planar tissue culture, electrospinning, and 3-dimensional printing.
(20) The system of any one of (1) to (19), wherein a length of the tubular support graft is less than a length of the tubular vascular graft.
(21) The system of any one of (1) to (20), wherein a length of the tubular support graft is greater than a length of the tubular vascular graft.
(22) The system of any one of (1) to (21), wherein a length of the tubular vascular graft is between 1 cm and 100 cm.
(23) An apparatus, comprising a tubular support graft comprising a 3-dimensional hydrogel matrix and cellular support structures, wherein the tubular support graft is configured to be concentrically positioned around a tubular vascular graft.
(24) The apparatus of (23), wherein the cellular support structures include at least one of a drug, a drug-releasing microparticle, and a drug-releasing nanoparticle.
(25) The apparatus of either (23) or (24), wherein the cellular support structures include at least one of a cell, an organoid, and a tissue.
(26) The apparatus of any one of (23) to (25), wherein the cell is a pancreatic islet cell.
(27) The apparatus of any one of (23) to (26), wherein the tubular support graft further comprises pancreatic islets.
(28) The apparatus of any one of (23) to (27), wherein the cellular support structures include at least one protein.
(29) The apparatus of any one of (23) to (28), wherein the cellular support structures include at least one of a vascular endothelial growth factor, a fibroblastic growth factor, a platelet derived growth factor, and a hepatocyte growth factor
(30) The apparatus of any one of (23) to (29), wherein the cellular support structures include at least one of a Fas ligand, and a nitric oxide donor.
(31) The apparatus of any one of (23) to (30), wherein the cellular support structures include endothelial cells.
(32) The apparatus of any one of (23) to (31), wherein the tubular support graft is formed by one of molding, casting, planar tissue culture, electrospinning, and 3-dimensional printing.
(33) The apparatus of any one of (23) to (32), wherein a length of the tubular support graft is less than a length of the tubular vascular graft.
(34) The apparatus of any one of (23) to (33), wherein a length of the tubular support graft is greater than a length of the tubular vascular graft.
(35) The apparatus of any one of (23) to (34), wherein a length of the tubular vascular graft is between 1 cm and 100 cm.
(36) A method for replacing a segment of vasculature, comprising providing a tubular support graft concentrically arranged against an outer surface of a support mandrel, contacting at least a planar end of the support mandrel with at least a planar end of a vascular graft mandrel, and sliding the tubular support graft over an outer surface of a tubular vascular graft concentrically arranged against an outer surface of the vascular graft mandrel.
(37) A method for forming a tubular support graft, comprising arranging a support mandrel within a tubular mold, a first end of the support mandrel and a first end of the tubular mold being fluidly sealed by a first end cap, injecting a mixture of hydrogel and cellular support structures within a volume formed between surfaces of the support mandrel and the tubular mold, fluidly sealing a second end of the support mandrel and a second end of the tubular mold via a second end cap, and removing the second end cap and the tubular sleeve after curing of the mixture to form the tubular support graft.
(38) A method for forming a tubular support graft, comprising injecting a mixture of hydrogel and cellular support structures into a culture dish, after the mixture cures, wrapping the cured mixture around a support mandrel, and securing open ends of the wrapped cured mixture to form the tubular support graft.
(39) A method for forming a tubular support graft, comprising rotating a support mandrel at a predetermined speed, depositing, via a 3-dimensional printer, droplets of a mixture of hydrogel and cellular support structures onto a surface of the rotating support mandrel, and after curing, removing the cured mixture from the rotating support mandrel to form the tubular support graft.
(40) An apparatus, comprising a tubular support graft comprising a 3-dimensional hydrogel matrix and cellular support structures, wherein the tubular support graft is configured to be concentrically positioned around a vessel segment harvested from patient vasculature.
(41) The apparatus of (40), wherein the cellular support structures include at least one of a drug, a drug-releasing microparticle, and a drug-releasing nanoparticle.

1(42) The apparatus of either (40) or (41), wherein the cellular support structures include at least one of a cell, an organoid, and a tissue.
(43) The apparatus of any one of (40) to (42), wherein the cell is a pancreatic islet cell.
(44) The apparatus of any one of (40) to (43), wherein the tubular support graft further comprises pancreatic islets.
(45) The apparatus of any one of (40) to (44), wherein the cellular support structures include at least one protein.
(46) The apparatus of any one of (40) to (45), wherein the cellular support structures include at least one of a vascular endothelial growth factor, a fibroblastic growth factor, a platelet derived growth factor, and a hepatocyte growth factor.
(47) The apparatus of any one of (40) to (46), wherein the cellular support structures include at least one of a Fas ligand, and a nitric oxide donor.
(48) The apparatus of any one of (40) to (47), wherein the cellular support structures include endothelial cells.
(49) The apparatus of any one of (40) to (48), wherein the tubular support graft is formed by one of molding, casting, planar tissue culture, electrospinning, and 3-dimensional printing.
(50) The apparatus of any one of (40) to (49), wherein a length of the tubular support graft is less than a length of the tubular vascular graft.
(51) The apparatus of any one of (40) to (50), wherein a length of the tubular support graft is greater than a length of the tubular vascular graft.
(52) The apparatus of any one of (40) to (51), wherein a length of the tubular vascular graft is between 1 cm and 100 cm.
(53) The apparatus of any one of (40) to (52), wherein the vessel segment is harvested from patient vasculature.
(54) A method for augmenting a segment of patient vasculature, comprising harvesting the segment of patient vasculature, sliding the harvested segment of patient vasculature over a vascular graft mandrel such that the harvest segment is concentrically arranged against an outer surface of the vascular graft mandrel, providing a tubular support graft concentrically arranged against an outer surface of a support mandrel, contacting at least a planar end of the support mandrel with at least a planar end of the vascular graft mandrel, and sliding the tubular support graft over an outer surface of the harvested segment to augment the harvested segment.
(55) A method for augmenting a segment of patient vasculature, comprising obtaining a vascular allograft, sliding the vascular allograft over a vascular graft mandrel such that the vascular allograft is concentrically arranged against an outer surface of the vascular graft mandrel, providing a tubular support graft concentrically arranged against an outer surface of a support mandrel, contacting at least a planar end of the support mandrel with at least a planar end of the vascular graft mandrel, and sliding the tubular support graft over an outer surface of the vascular allograft to augment the vascular allograft.

1. A system for replacement of a segment of vasculature, comprising:
a tubular vascular graft concentrically arranged against an outer surface of a first mandrel; and
a tubular support graft concentrically arranged against an outer surface of a second mandrel, the tubular support graft being slidable over an outer surface of the tubular vascular graft,
wherein an outer diameter of the second mandrel and an outer diameter of the tubular vascular graft are sized such that the tubular support graft can slide over the outer surface of the tubular vascular graft when the segment of the vasculature is replaced.

2. The system of claim 1, wherein the tubular vascular graft is a construct comprising a 3-dimensional proteinaceous extracellular matrix, an autograft, an allograft, or a synthetic graft.

3. The system of claim 1, wherein the tubular vascular graft is a construct comprising a 3-dimensional proteinaceous extracellular matrix subjected to decellularization, and wherein the construct was formed by seeding a substrate with cells and maintaining the cells under conditions suitable for growth of the cells, whereby the proteinaceous extracellular matrix is formed around the cells.

4. The system of claim 3, wherein the substrate is polyglycolic acid.

5. The system of claim 3, wherein the 3-dimensional proteinaceous extracellular matrix comprises collagen.

6. The system of claim 1, wherein the tubular vascular graft comprises smooth muscle cells.

7. The system of claim 1, wherein the tubular vascular graft comprises endothelial cells.

8. The system of claim 1, wherein the tubular vascular graft comprises smooth muscle cells toward the outer surface of the tubular vascular graft and endothelial cells toward an inner surface of the tubular vascular graft.

9. The system of claim 1, wherein the tubular vascular graft comprises autologous cells.

10. The system of claim 1, wherein the tubular support graft comprises a 3-dimensional hydrogel matrix and cellular support structures.

11. The system of claim 10, wherein the cellular support structures include at least one of a drug, a drug-releasing microparticle, and a drug-releasing nanoparticle.

12. The system of claim 10, wherein the cellular support structures include at least one of a cell, an organoid, and a tissue.

13. The system of claim 12, wherein the cell is a pancreatic islet cell or an induced pluripotent stem cell-derived pancreatic islet cell.

14. The system of claim 10, wherein the tubular support graft further comprises pancreatic islets.

15. The system of claim 10, wherein the cellular support structures include at least one protein.

16. The system of claim 10, wherein the cellular support structures include at least one of a vascular endothelial growth factor, a fibroblastic growth factor, a platelet derived growth factor, and a hepatocyte growth factor.

17. The system of claim 10, wherein the cellular support structures include at least one of a Fas ligand, and a nitric oxide donor.

18. The system of claim 10, wherein the cellular support structures include endothelial cells.

19. The system of claim 1, wherein the tubular support graft is formed by one of molding, casting, planar tissue culture, electrospinning, and 3-dimensional printing.

20. The system of claim 1, wherein a length of the tubular support graft is less than a length of the tubular vascular graft.

21. The system of claim 1, wherein a length of the tubular support graft is greater than a length of the tubular vascular graft.

22. The system of claim 1, wherein a length of the tubular vascular graft is between 1 cm and 100 cm.

23. An apparatus, comprising:
a tubular support graft comprising a 3-dimensional hydrogel matrix and cellular support structures, wherein the tubular support graft is configured to be concentrically positioned around a tubular vascular graft.

24. The apparatus of claim 23, wherein the cellular support structures include at least one of a drug, a drug-releasing microparticle, and a drug-releasing nanoparticle.

25. The apparatus of claim 23, wherein the cellular support structures include at least one of a cell, an organoid, and a tissue.

26. The apparatus of claim 25, wherein the cell is a pancreatic islet cell or an induced pluripotent stem cell-derived pancreatic islet cell.

27. The apparatus of claim 23, wherein the tubular support graft further comprises pancreatic islets.

28. The apparatus of claim 23, wherein the cellular support structures include at least one protein.

29. The apparatus of claim 23, wherein the cellular support structures include at least one of a vascular endothelial growth factor, a fibroblastic growth factor, a platelet derived growth factor, and a hepatocyte growth factor.

30. The apparatus of claim 23, wherein the cellular support structures include at least one of a Fas ligand, and a nitric oxide donor.

31. The apparatus of claim 23, wherein the cellular support structures include endothelial cells.

32. The apparatus of claim 23, wherein the tubular support graft is formed by one of molding, casting, planar tissue culture, electrospinning, and 3-dimensional printing.

33. The apparatus of claim 23, wherein a length of the tubular support graft is less than a length of the tubular vascular graft.

34. The apparatus of claim 23, wherein a length of the tubular support graft is greater than a length of the tubular vascular graft.

35. The apparatus of claim 23, wherein a length of the tubular vascular graft is between 1 cm and 100 cm.

36. A method for replacing a segment of vasculature, comprising:
providing a tubular support graft concentrically arranged against an outer surface of a support mandrel;
contacting at least a planar end of the support mandrel with at least a planar end of a vascular graft mandrel; and
sliding the tubular support graft over an outer surface of a tubular vascular graft concentrically arranged against an outer surface of the vascular graft mandrel.

37-55. (canceled)

56. The system of claim 10, wherein the tubular support graft further comprises a polymer for structural reinforcement.

57. The system of claim 56, wherein the polymer comprises at least one of poly(glycolic acid) (PGA), poly(lactic acid) (PLA), poly(lactic-co-glycolic acid) (PLGA), polyethylene terephthalate (PET), polycaprolactone (PCL), poly(lactide-co-caprolactone) (PLCL), poly(methyl methacrylate) (PMMA), poly(vinyl alcohol) (PVA), poly(ethylene glycol) (PEG) polytetrafluoroethylene (PTFE), expanded polytetrafluoroethylene (ePTFE), polyurethane (PU), poly(glycerol-co-sebacate) (PGS), and poly(ethylene glycol) diacrylate (PEGDA).

58. The apparatus of claim 23, wherein the tubular support graft further comprises a polymer for structural reinforcement.

59. The apparatus of claim 58, wherein the polymer comprises at least one of poly(glycolic acid) (PGA), poly(lactic acid) (PLA), poly(lactic-co-glycolic acid) (PLGA), polyethylene terephthalate (PET), polycaprolactone (PCL), poly(lactide-co-caprolactone) (PLCL), poly(methyl methacrylate) (PMMA), poly(vinyl alcohol) (PVA), poly(ethylene glycol) (PEG) polytetrafluoroethylene (PTFE), expanded polytetrafluoroethylene (ePTFE), polyurethane (PU), poly(glycerol-co-sebacate) (PGS), and poly(ethylene glycol) diacrylate (PEGDA).
